(19) United States
(12) Patent Application Publication
(10) Pub. No.: US 2019/0380761 A1
(43) Pub. Date: Dec. 19, 2019

(54) CONTROLLING ESOPHAGEAL TEMPERATURE DURING CARDIAC ABLATION

(71) Applicant: University of Florida Research Foundation, Inc., Gainesville, FL (US)

(72) Inventors: John N. Catanzaro, Jacksonville, FL (US); Michele N. Dill, Gainesville, FL (US); Christopher D. Batich, Gainesville, FL (US)

(21) Appl. No.: 16/553,479

(22) Filed: Aug. 28, 2019

Related U.S. Application Data
(63) Continuation-in-part of application No. PCT/US2018/001941, filed on Feb. 23, 2018.
(60) Provisional application No. 62/464,653, filed on Feb. 28, 2017.
(60) Provisional application No. 62/538,022, filed on Jul. 28, 2017.
(60) Provisional application No. 62/746,739, filed on Oct. 17, 2018.

(51) Int. Cl. A61B 18/00

(52) U.S. Cl. CPC A61B 18/00; A61B 2018/00029; A61B 2018/0212; A61B 2018/00351; A61B 2018/00577; A61B 2018/00285

(57) ABSTRACT

A flexible catheter is inserted into the esophagus to cool or warm the esophagus, particularly during certain procedures which can tend to change the temperature in the area of the esophagus. The catheter is inserted through the mouth and throat to a position, for example, proximate the heart, but within the esophagus. A thermally conductive gel is injected into the esophagus where it is immobilized by the one or more balloons. A coolant is pumped through a coolant tube affixed to the catheter, where it exchanges heat with the conductive gel.

### CROSS-REFERENCE TO RELATED APPLICATION

This application is a continuation-in-part application of and claims priority PCT Application No. PCT/US2018/019410, filed Feb. 23, 2018, which claims priority to and the benefit of U.S. Provisional Application No. 62/464,653, filed Feb. 28, 2017 and U.S. Provisional Application No. 62/538,022, filed Jul. 28, 2017, these disclosures of which are hereby incorporated by reference in their entireties, including all figures, tables and drawings.

This application also claims priority to and the benefit of Provisional Application No. 62/746,739, filed Oct. 17, 2018, the application of which is incorporated by reference in its entirety.

### FIELD OF THE DISCLOSURE

The disclosure relates to a system and method for controlling esophageal temperature during cardiac ablation, and in particular, to changing the temperature in an interior of the esophagus.

### BACKGROUND OF THE DISCLOSURE

Ablation of tissues surrounding the pulmonary veins is carried out to disrupt an electrical signal transmitted from the veins into the left atrium, giving rise to atrial fibrillation. One technique for creating this ablation is the Convergent Procedure, which uses radio frequency energy to generate heat which is applied to heart tissue to produce ablation and interrupt the signal.

Radiofrequency ablation, specifically left atrial endocardial ablation or pulmonary vein isolation in patients with symptomatic paroxysmal or persistent atrial fibrillation uses radiofrequency energy applied to the left atrium at the ostium of the pulmonary veins and sometimes on the posterior wall. An atrial esophageal fistula is a known and debilitating (if not fatal) complication resulting in fistula formation between the atrium and esophagus with entry of air into the left atrium. This may lead to cerebrovascular attack and or myocardial infarction. In addition to standard pulmonary vein isolation, the Convergent Procedure is generally performed in patients with symptomatic persistent atrial fibrillation. An initial part of the procedure utilizes a radio frequency (RF) probe or coil which is placed transdiaphragmatically on an exterior surface of the heart on the posterior wall of the epicardium, in an effort to ablate the epicardial posterior wall. The device utilizes RF energy emitted from a generator which is grounded to the patient. A coil apparatus is introduced telescopically onto the epicardium which then uses a vacuum suction while applying the RF energy. The impedance is measured while RF is applied in an effort to confirm that the application of energy is complete, and that sufficient energy has been transmitted to the epicardium in order to cause ablation.

To complete a desired ablation pattern near the blood vessels, ablation is additionally performed inside the heart using electrophysiology. A device is threaded through the femoral artery into the heart, and RF energy is again used to complete portions of the ablation pattern which could not be completed outside the heart.

Cryothermal energy has been used inside the heart on the endocardium to ablate the ostium of pulmonary veins, including for example by use of the ARTIC FRONT device of Medtronic, Inc. The device occludes the ostium with a round balloon-like structure which is inserted into the ostium to make contact with body tissue, and which is then filled with a coolant to cause freezing of tissue at the ostium.

Laser ablation has also been used to isolate the pulmonary veins in symptomatic paroxysmal atrial fibrillation via an endoscopic balloon introduced transseptally into the left atrium. The probe is placed into the pulmonary vein and the balloon is deployed giving the operator visualization of the pulmonary vein before applying laser application. Laser application can increase left atrial temperature and predispose the esophagus to collateral damage via thermal injury

All of the above modalities have a latent effect of energy, that is, when stopping radiofrequency, or laser, the temperature measured in the esophagus continues to rise to a plateau before nadir. Cryothermal may have the same effect but in an opposite direction “freeze”.

### SUMMARY OF THE DISCLOSURE

Aspects of the present disclosure are related to apparatuses and methods for cooling or warming an interior area of the esophagus during a therapeutic procedure.

In one aspects, among others, a device for cooling or warming an interior area of an esophagus during a therapeutic procedure, comprises a flexible tube having a proximal end and a distal end, the flexible tube being passable from outside of the body to the interior area of the esophagus and including at least one gel passing port formed through the tube; a coolant tube affixed to an exterior surface of the flexible tube, the coolant tube extending from the proximal end of the flexible tube to the distal end of the flexible tube; and at least one balloon affixed to the exterior surface of the flexible tube, the at least one balloon being configured to block the esophagus when inflated to prevent a gel released through the at least one substance passing port from entering another area of the body.

In various aspects, the device further includes at least one temperature sensor positioned along a length of the tube and configured to output temperature information pertaining to a plurality of areas of the esophagus. In various aspects, the tube forms at least one bend whereby the tube is passable back outside of the body, the tube thereby forming two ends both outside of the body, the one or more tube ports positioned proximate the interior area of the esophagus. In various aspects, the device further comprises a flexible sleeve slidable in connection with the tube and including at least one substance passing port formed through the sleeve, the sleeve sized with respect to the tube to form a tight seal with the tube such that when at least one substance passing port of the sleeve is aligned with the at least one substance passing port of the tube, a substance may pass through the sleeve and the tube. In various aspects, the sleeve is slidable within the tube. In various aspects, the sleeve is slidable along an exterior of the tube. In various aspects, a distal end of the flexible tube is passed first into the body and is surrounded by an outer tube which captures liquid which has passed through a flexible sleeve. In various aspects, a distal end of the flexible tube that is passed first into the body is surrounded by the at least one balloon which captures liquid which has passed through the flexible sleeve. In various aspects, the coolant tube is formed into a coil.

In various aspects, a kit comprising the device and the gel. In various aspects, the gel comprises water and a polyalkylene glycol. In various aspects, the polyalkylene glycol comprises polyethylene glycol, polypropylene glycol, monomethoxy polyethylene glycol, a poloxamer, or any combination thereof. In various aspects, the polyalkylene glycol has a molecular weight of about 600 Da to about 6,000 Da. In various aspects, the polyalkylene glycol is from about 0.1 wt % to 5 wt % of the gel. In various aspects, the gel has a dielectric constant of less than 20. In various aspects, the gel comprises a thermally conductive gel.

In other aspects, among others, a method for cooling or warming an interior area of the esophagus during a therapeutic procedure comprises inserting a temperature-cooling device into the esophagus; inflating at least one balloon of the device to block at least one section of the esophagus; and injecting a therapeutic substance into a therapeutic substance lumen of the device in order to deposit the therapeutic substance into the esophagus, the at least one balloon blocking the therapeutic substance from traveling to other areas of the body.

In various aspects, the therapeutic substance comprises water and a polyalkylene glycol and the polyalkylene glycol comprises polyethylene glycol, polypropylene glycol, monomethoxy polyethylene glycol, a poloxamer, or any combination thereof. In various aspects, the polyalkylene glycol has a molecular weight of about 600 Da to about 6,000 Da. In various aspects, the polyalkylene glycol is from about 0.1 wt % to 5 wt % of the gel.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 depicts a diagrammatic cross-sectional view of the esophagus and left atrium, and an example of a temperature controlling device which is releasing a liquid at a desired cooling or warming temperature, from a selected port, onto an interior surface of the esophagus, according to various embodiments of the present disclosure.

FIG. 1A depicts an example of an alternative temperature controlling device, in which ports are formed by an external sliding sleeve, according to various embodiments of the present disclosure, in which ports are formed by an external sliding sleeve.

FIG. 2 depicts an example of a proximal end of a temperature controlling device, and deployment within the body, according to various embodiments of the present disclosure.

FIG. 3 depicts a multilumen configuration of the device according to various embodiments of the present disclosure.

FIG. 4 depicts an example of an alternative device of the disclosure, also releasing a liquid upon an interior surface of the esophagus, the liquid retrieved using an aspiration channel, according to various embodiments of the present disclosure.

FIG. 5 depicts an example of a temperature controlling device, without the aspiration channel, according to various embodiments of the present disclosure.

FIG. 6 depicts an example of a temperature controlling device, connected to a steerable device, according to various embodiments of the present disclosure.

FIG. 7 depicts an example of an alternative temperature controlling device of the disclosure in which a warming or cooling liquid is passed through the device within an interior of the esophagus, the liquid not being released into the esophagus, according to various embodiments of the present disclosure.

FIG. 8 depicts an example of an alternative temperature controlling device of the disclosure in which a warming or cooling liquid is passed through the device within an interior of the esophagus, the liquid being released into a flexible balloon, and including an aspiration channel disposed within the balloon, according to various embodiments of the present disclosure.

FIG. 9 depicts an example of an alternative temperature controlling device of the disclosure in which a warming or cooling liquid is circulated through separate coils positioned within an interior of the esophagus, according to various embodiments of the present disclosure.

FIG. 10 depicts an example of an alternative device of the disclosure in which a substance is discharged inside of a tube placed within the esophagus, according to various embodiments of the present disclosure.

FIG. 11 depicts the tube of FIG. 10, further including a core extension which elutes or releases a therapeutic substance, according to various embodiments of the present disclosure.

FIG. 12 depicts an alternative embodiment of the disclosure in which a therapeutic substance is introduced into a balloon which expands and can release the substance through pores, according to various embodiments of the present disclosure.

FIG. 13 depicts the balloon of FIG. 12, further including a core extension which elutes or releases a therapeutic substance, according to various embodiments of the present disclosure.

FIG. 14 depicts an embodiment of the disclosure including an open loop/closed loop system with an eluting core, according to various embodiments of the present disclosure.

FIG. 15 depicts a device of the disclosure including an expandable sponge, the sponge in a contracted state, according to various embodiments of the present disclosure.

FIG. 16 depicts the device of FIG. 15, the sponge in an expanded state, filled with an expandable material, according to various embodiments of the present disclosure.

FIG. 17 depicts a device of the disclosure including a balloon filled with an expandable material, the balloon in a deflated or partially deflated state, according to various embodiments of the present disclosure.

FIG. 18 depicts the device of FIG. 17, the balloon fully inflated, according to various embodiments of the present disclosure.

FIG. 19 depicts an example of a device for cooling the esophagus during an atrial ablation according to various embodiments of the present disclosure.

FIG. 20 depicts another example of a device for cooling the esophagus during an atrial ablation according to various embodiments of the present disclosure.

FIG. 21 depicts another example of a device for cooling the esophagus during an atrial ablation according to various embodiments of the present disclosure.

FIG. 22 depicts another example of a device for cooling the esophagus during an atrial ablation according to various embodiments of the present disclosure.

FIG. 23 depicts a device of the disclosure for mimicking the spatial relationship and thermal conductivity of the left atrium and esophagus according to various embodiments of the present disclosure.

FIG. 24 is an example of a graph showing change in temperature at the esophageal wall of an in vitro testing platform mimicking esophageal temperature during atrial ablation according to various embodiments of the present disclosure.

FIG. 25 is an example of a graph showing change in temperature at the esophageal wall as in FIG. 26 using a device of the disclosure for cooling the esophagus during an atrial ablation according to various embodiments of the present disclosure.

FIG. 26 is an example image of a device for cooling the esophagus during an atrial ablation according to various embodiments of the present disclosure.

FIG. 27 is an example image of a device for cooling the esophagus during an atrial ablation according to various embodiments of the present disclosure.

FIG. 28 depicts a wireless communication device, some or all of which can be used in carrying out the disclosure.

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

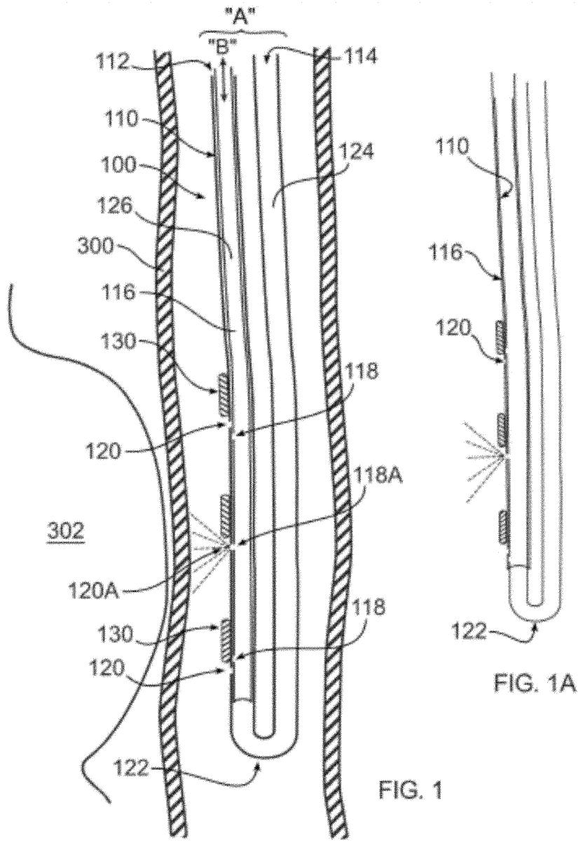

In accordance with the disclosure, open or closed loop irrigation of cooling or warming liquid is applied to an interior surface of the esophagus that is proximate to the heart during a procedure which is applying heat or cold to the heart, and particularly in the region of the left atrium 302 (FIG. 1) which is most proximate the esophagus. Such procedures can include ablation of the heart using heat, such as during radio frequency or laser ablation, or ablation of the heart using cooling, such as during cryoablation. A device of the disclosure is used to apply a cooling liquid to an interior surface of the esophagus during heat ablation, and a warming liquid during cold ablation. The applied liquid acts as a medium to counterbalance a resultant and undesired change in temperature of the esophagus, which can otherwise damage the esophagus during the therapeutic treatment of the heart. Such damage can include the formation of ulcers or an esophageal fistula, for example. The disclosure provides medical devices that improve the safety of left atrial ablation by reducing the incidence and severity of esophageal injury, by providing for targeted temperature control.

The apparatuses of the present disclosure can also help the electrophysiologist or cardiothoracic surgeon gain information on which types of lesion orientation from the catheter (sliding, parallel or perpendicular can afford a transmurality lesion while preserving the integrity of the esophagus.

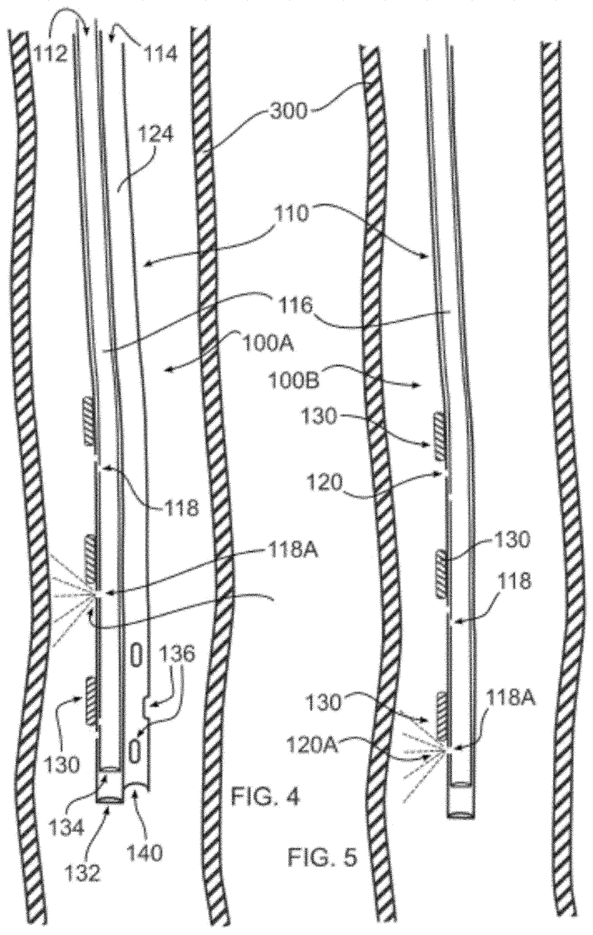

The disclosure provides closed, semi-closed, and open loop irrigation devices and methods. As discussed further below, closed loop devices, such as are shown in FIGS. 7 and 9-11, do not release any liquids into the esophagus. Semi-closed devices, such as is shown in FIG. 1, do release liquid at predetermined temperatures to directly contact the esophagus with the liquid at a desired treatment site, but recover all remaining liquid that has not been released. Open loop devices, such as are shown in FIGS. 4-6, additionally release liquids at locations other than a desired treatment site, although the devices of FIGS. 4 and 5 attempt to aspirate such released liquids using suction.

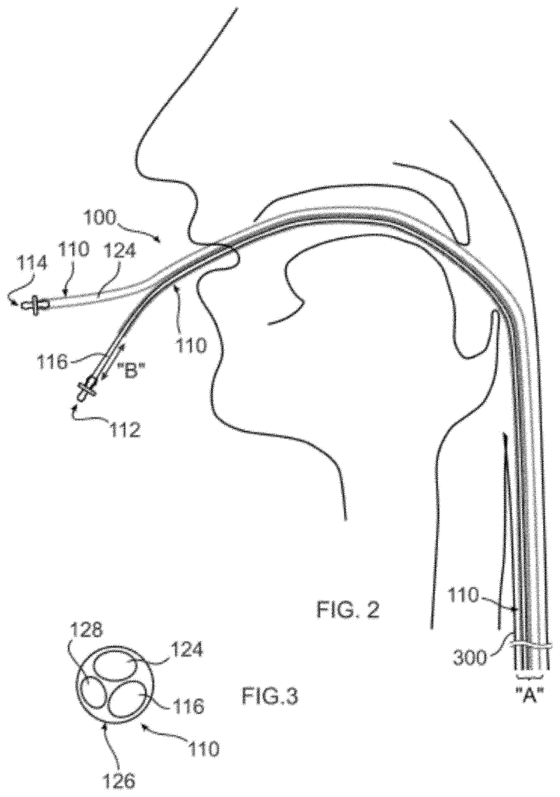

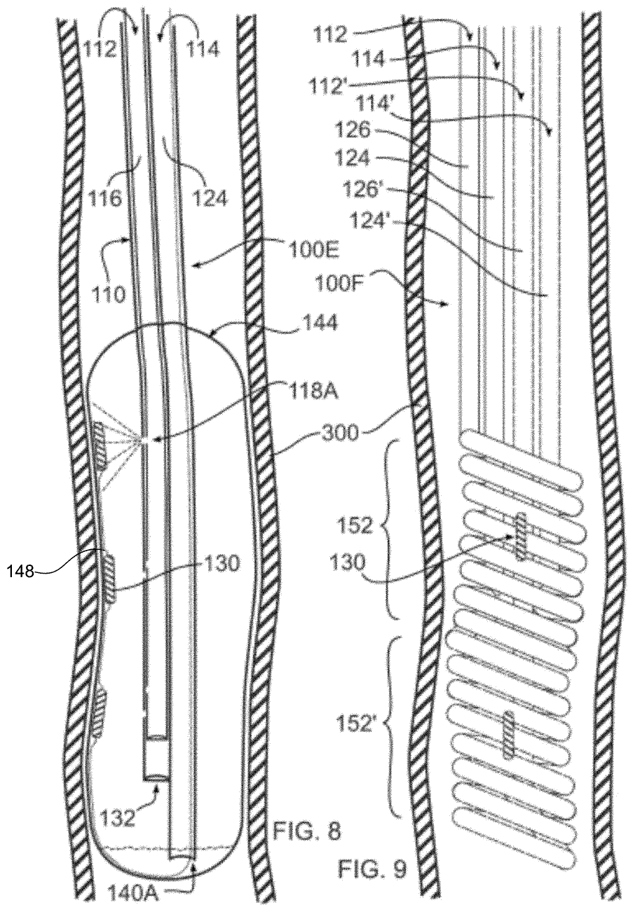

With reference to FIG. 1, a device 100 of the disclosure includes a tube 110 which passes into the esophagus forming an inlet 112 and a supply channel 126, at least one bend 122, and an outlet 114 disposed at the end of a return channel. A sleeve 116 is disposed within tube 110 and includes a plurality of ports 118. Tube 110 is illustrated as a linear channel with a single loop or bend 122, for clarity; however, it should be understood that tube 110 can be formed in the shape of a coil, such as is illustrated in FIG. 8, or in another shape or pattern. For example, tube 110 can have the shape of a closed ended sleeve, e.g. a test-sleeve shape, with interior channels connected to inlet 112 and outlet 114. An upper portion indicated by brace “A” continues up and out of the esophagus, as illustrated in FIG. 2.

Interiorly disposed slidable sleeve 116 passes through a portion of an interior of tube 110, and includes a plurality of ports 118 mutually spaced apart along a length of sleeve 116, and which open a passage an interior of sleeve 116 to an exterior of sleeve 116. Sleeve 116 is sized to form a liquid tight seal against an interior of tube 110. Mating ports are provided along tube 110, whereby when a port 118 is aligned with a port 120, liquid within sleeve 116 can be released or sprayed from tube 110, where it may contact an adjacent area of esophagus 300 to either cool or warm such area. In FIG. 1, a second or middle port 120A of tube 110 is aligned with aligned port 118A of sleeve 116. The relative spacing of ports 118 is selected to align only one mating port set of port 118 and 120 at the same time. Other alignments of ports 118 and 120 can be configured where more than one mating port pair are opened simultaneously. While sleeve 116 is illustrated as positioned along supply channel 126, it can also or alternatively be positioned, together with ports 120, along return channel 124.

Port openings can have a diameter of, for example, 0.4 mm, although they may be much smaller or larger, for example 0.01 mm to 5 mm. For smaller diameters, or other configurations in which a potentially undesirable build-up of pressure can take place within the body, it can be advantageous to provide a pressure relief valve outside of the body.

As additionally shown in FIG. 1, a temperature sensor 130 is positioned at one or more location along tube 110. Sensor 130 can transmit temperature data corresponding to an adjacent area along the esophagus 300. A plurality of sensors can provide temperature information for a plurality of areas along esophagus 300, whereby signal processing equipment connected to sensors 130 can identify particular areas of the esophagus which are experiencing or anticipated to experience an undesired temperature change. Sensors 130 can transmit this data via wires 148 (FIG. 8), or by a wireless communication, such as WIFI, BLUETOOTH or other nearfield protocol, and any other wireless protocol. To avoid inadvertent heating of sensor 130 by radiofrequency associated with RF ablation, it can be advantageous to avoid using metal in sensor 130. This may be achieved using a fiber optic sensor, for example. Other non-metallic or metallic temperature sensor technologies may be used for sensor 130.

In an embodiment, an electronic processor 802 receives temperature information from sensors 130, and causes movement of sleeve 116 to aligns ports 118 and 120 at one or more locations most proximate to a sensor reporting an undesired esophageal temperature.

Alternatively, a medical practitioner can view analog or digital readouts of temperature sensors 130, and can compare reference information of port locations with the sensor 130 reporting an undesired temperature, and using indicia (not shown) placed upon tube 110 and sleeve 116, slide sleeve 116 relative to tube 110 to align desired ports for release of temperature stabilizing liquid proximate an area of undesired esophageal temperature.

FIG. 1A illustrates that sleeve 116 can pass over an outside of tube 110, to thereby expose ports 118 and 120 as described above, and in other respects the device of FIG. 1A functions as described herein. Devices 100 A-C and E can be similarly adapted.

FIG. 2 diagrammatically depicts device 100 deployed within the body, passing from outside the body into the esophagus 300. While slidable sleeve 116 and return channel 124 appear as separate tubes, as shown in FIG. 3, it should be understood that they may be formed as lumens within a common or multilumen catheter tube 110. Other lumens 128 may be provided within catheter tube 110, through which other materials may be passed, such as electrical wires, additional sensors, surgical instruments, gases, or as further described herein, steerable devices. FIG. 2 illustrates one method of passing a fluid into slidable sleeve 116, while enabling sleeve 116 to be moved within tube 110, and to receive unreleased or recaptured fluid from outlet 114. It would be understood within the art that there are other methods for separately admitting or receiving fluids or materials through the various lumen of a multilumen catheter.

With reference to FIG. 4, in device 100A, bend 122 is not present, and the distal ends 132, 134 of tube 110 and slidable sleeve 116, respectively, are sealed. Return channel 124 is provided with one or more aspiration ports 138, and outlet 114 is connected to a source of vacuum, whereby fluids released adjacent to the ports 138 are aspirated out of esophagus 300 after contacting esophagus 300. A distal end 140 can be open and serve as an aspiration port, or it may be closed. As such, fluid may be admitted into tube 110 and slidable sleeve 116 at a lower pressure than for device 100 for a similar spray velocity at active port 118A/120A. While recovery of warming/cooling fluid may not be as efficient as for device 100, it may be sufficient depending on the total amount of fluid to be released, and the effect of the released fluid upon the esophagus or other components of the digestive system. If it is not necessary to recover fluid, device 100B of FIG. 5 can be used, which is similar to device 100A, but omits return channel 124 and associated ports 120.

In accordance with the disclosure, suction can be applied to housing 110, exposed through ports 1136, or ports formed by distal ends 140, 140A, or other port, which can be used to attach or draw device 100 towards selected areas of body tissue. A separate channel can be provided for this purpose.

In FIG. 4, active ports 118A/120A are aligned to illustrate release from a second port, and in FIG. 5, active ports 118A/120A are aligned to illustrate release from a third, or lowest port. The selection of port for the illustration is arbitrary, however, and it should be understood that any port may be activated for either device 100A/100B, as well as for other devices of the disclosure, as described herein.

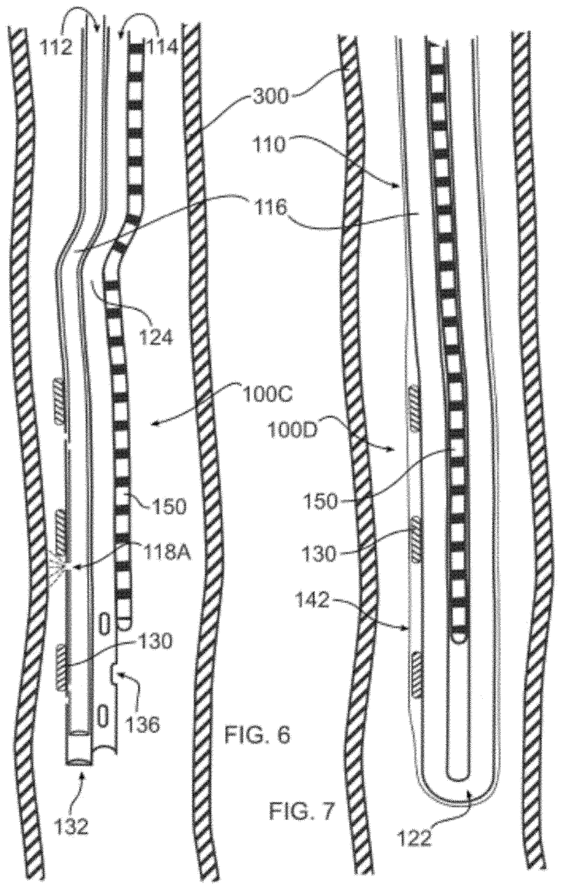

In FIG. 6, device 100C includes the components of device 100A of FIG. 4, and further includes a steerable device 150, such as a steerable catheter or other device which enables distal manipulation in the manner of a steerable catheter (e.g. with or without an associated lumen), connected along all or part of its length to sleeve 116 and channel 124, whereby as steerable device is manipulated to bend as is known in the art, remaining components of device 100C are similarly manipulated to be moved within esophagus 300. For example, device 100C can be moved by the steerable device to be closer or farther from an area indicated by a sensor 130 that has an undesired temperature. It is noted, however, that there is some clinical evidence that it may be undesirable to forcibly press a wall of esophagus during ablation, particularly if such action moves tissue of the esophagus closer to left atrium 302, other otherwise closer to a region of ablation. Steerable device can be configured to extend out of the mouth of the patient in order to be manipulated, as understood within the art.

As shown in FIG. 7, device 100D includes the components of device 100C and steerable device 150 as described with respect to FIG. 6, and further includes an external flexible housing 142 surrounding all components, extending along all or a portion of a length of the components. While the device of FIG. 1 is illustrated within housing 142, it should be understood that any of the devices of the disclosure, for example including any of devices 100A-F, can be entirely or partially enclosed by housing 142, and/or can include steerable device 150, as described herein. Housing 142 facilitates inserting and removing devices of the disclosure by forming a smooth exterior surface, and can be fabricated with a material that is non-reactive and non-allergenic, to isolate housed components from contact with body tissue, thereby facilitating the selection of materials for the housed components.

In FIG. 8, components at a distal end of device 100E of the disclosure are enclosed within a flexible housing 144 which can be semi-rigid or inflatable. Housing 144 is sized, configured, or is inflatable to conform to an interior perimeter of esophagus 300. Housing 144 can be fabricated using a flexible polymer or other biocompatible material of predetermined shape, that is sufficiently flexible to be reduced in size during deployment to be passable to the treatment site within the esophagus. Alternatively, housing 144 can include a flexible outer skin, and further include a resilient internal scaffold, for example of shape memory alloy, which is reversibly collapsible. As a further alternative, housing 144 can be formed as an inflatable balloon, where pressurized air is passed through tube 110, or another lumen of a multilumen catheter, as described herein. In each variation, housing 144 can expand or is expandable to contact the inner surfaces of the esophagus when positioned at a selected location along the esophagus.

Once positioned, device 100E can be used as otherwise described for the various embodiments of device 100 described herein which release, emit, or spray a fluid from aligned ports 118 A/120 A. However, housing 144 is fabricated with an outer material which will not pass a warming/cooling fluid outside housing 144. Accordingly, as an outer material of housing 144 is cooled or warmed, it will cool or warm esophageal tissue which it contacts, thereby providing the therapeutic benefit described herein of controlling the temperature of an area of the esophagus. FIG. 8 depicts an open loop fluid retrieval system; however, housing 144 can be used with the partially open or closed systems described herein. While housing 144 is illustrated as only being present at a distal end of device 100E, housing 144 can continue proximally along any length of device 100E, whether or not ports 120 extend along such lengths.

In device 100E, distal end 140A of return channel 124 is positioned proximate a lower end of housing 144, to be positioned within accumulated warming/cooling fluid which may then be aspirated. As such, ports 136 are not necessary, although they may be provided to increase capacity. Wires 148 communicating from sensors 130 to outside of the body are illustrated passing through channel 124, although they may pass along another route, as detailed with respect to FIG. 3.

FIG. 9 depicts device 100F of the disclosure, in which one or more coils 152, two of which are illustrated as 152 and 152′, each providing a circuit for the flow of cooling/warming liquid. The coils include tubes 110 which can include slidable sleeves 116, but, in this embodiment, do not. More particularly, coils 152 can be disposed with respect to each other, so that each coil is adjacent to a different area of the interior of esophagus 130. In the embodiment shown, coils 152 are positioned vertically or successively disposed with respect to each other, although coils 152 can additionally or alternatively be placed side by side with respect to each other. In this manner, fluid can be directed preferentially or solely through a coil which is proximate to an area of esophagus which is expected to be experiencing an undesired temperature change, or which is actually experiencing such temperature change as indicated by sensors 130, as described elsewhere herein. Each coil 152 is formed as a tube 110 which includes a supply channel 126 and a return channel 124. As shown, the channels can be nested within the coil to produce a device of smaller diameter. In an embodiment, these channels can be connected to valves (not shown) positioned inside or outside of the body, which valves may be controlled in a known manner to select a particular coil or coils for controlled amounts of fluid flow.

Coils 152 can be placed within an outer sleeve or housing, not shown, to provide further structural integrity and to ease insertion and removal from the body. In an embodiment, a sleeve can be slid over coils 152 or portions of coils 152, to either cover and insulate them, or to selectively expose coils to desired portions of the esophagus. As with other embodiments herein, a steerable device 150 can be associated with coils 152 so that they can be therapeutically located laterally within the esophagus, either closer or farther from an area of undesired esophageal temperature.

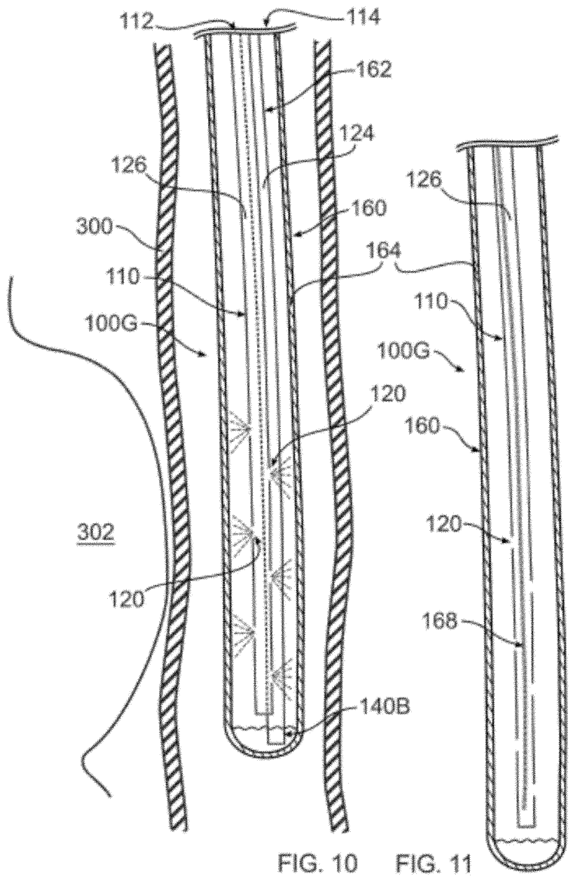

Turning now to FIG. 10, a device 100G of the disclosure includes a flexible closed ended tube 160 which forms a housing to contain a tube 110 having one or more ports 120. A cooled or heated liquid or gel is passed into tube 110 via supply channel 126, to be discharged at ports 120 against an inner surface of tube 110. As such, tube 110 is cooled or heated proximate the area or areas of discharge, to thereby change a temperature of the esophagus 300 adjacent to the areas of discharge.

Fluid that has been discharged collects at the bottom of tube 160, where it may remain during a therapeutic procedure, or where it may be aspirated, for example using a return tube 162, having a distal opening 140B near a distal end of tube 160. The discharged fluid is drawn through return channel 124, where it may again be cooled or heated and reused, or it may be discarded.

Tube 162 is depicted as a separate tube that is viewed partially behind tube 110 in FIG. 10, although tube 162 can be realized in other known forms, such as a lumen associated with tube 160, for example. Tube 162 may also be inserted within tube 160 when the level of discharged fluid is known to be of a predetermined volume, and can be removed after the fluid is aspirated.

Tube 160 is shown with a coating 164 which can include any or all of a lubricious coating as known in the art to facilitate insertion into the esophagus; a therapeutic substance; and a substance, such as a gel, which can be heated or cooled to therapeutically treat the esophagus.

A therapeutic substance can include a material to reduce acid, and the effects of acid, in the area of treatment, for example a slurry of coating solution containing calcium carbonate or other acid neutralizing substance, or an agent which reduces the formation of acid in the digestive tract, including for example omeprazole, such as omeprazole magnesium. The eluting substance, or the coating upon tube 160, can be any other therapeutically beneficial substance or combination of substances, including for example agents which promote healing, antimicrobial agents, or drugs to treat a disease condition of the patient, including for example a drug to treat a condition of the esophagus or heart. Coating 164 can additionally be applied to the outer surface of other embodiments 100-100Q, described herein, which may contact the esophagus.

FIG. 11 depicts device 100G, as well, although tube 162 has been removed for clarity. In FIG. 11, a drug eluting core extension 168 extends within supply channel 126 of tube 110, whereupon a therapeutic substance 166 is released as fluid flows through channel 126. In the embodiment shown, extension 168 has the form of an elongate rod, although other shapes can be used, such as a coil or lozenge. Additionally, extension 168 can be positioned outside of tube 110, and within tube 160, whereupon it will release the therapeutic substance when discharge through ports 120 occurs. The therapeutic substance 166 can be combined with a binder, and coated upon extension 168, to release the substance 166 upon contact with a liquid, such as water, as is understood within the art. Alternatively, the therapeutic substance can be incorporated into material which forms extension 168, to elute from extension 168, or to be released as extension 168 is dissolved by the heating or cooling liquid to be discharged. The therapeutic substance can be, for example, any of the substances listed herein with respect to coating 164. Extension 168 can be formed with a colloid, gel, sol, or emulsion, which dissolves to release, or otherwise releases the therapeutic substance in the body over time. Alternatively, extension 168 can be formed as a thermopolymer or other natural or synthesized substrate which elutes a substance when hydrated by an introduced fluid or by body fluid, or when warmed by the body.

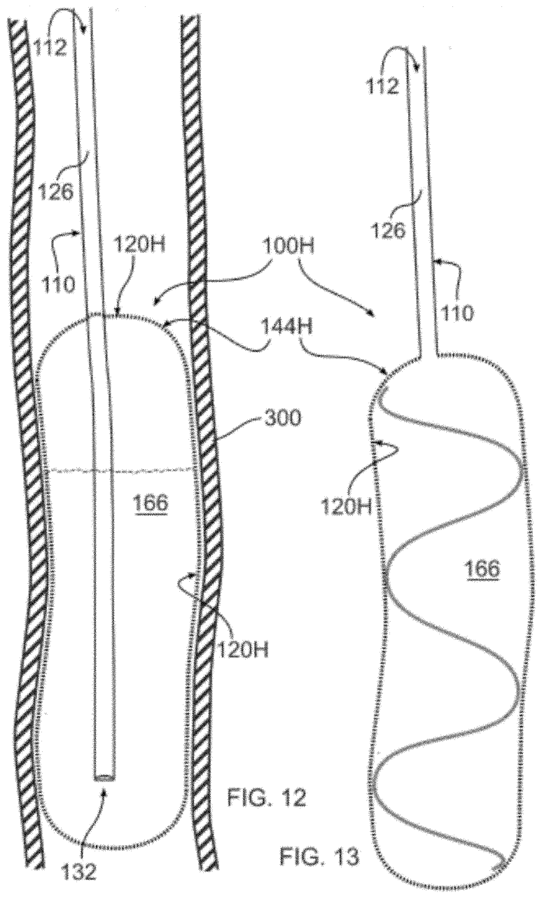

With reference to FIGS. 12 and 13, a device 100H of the disclosure includes an expandable portion or balloon 144H which is in fluid communication with a tube 110 and supply channel 126. Balloon 144H includes a plurality of pores 120H through which a substance 166 introduced into supply channel 126 can flow to pass into contact with an inner surface of esophagus 300. Pores 120H are sized and provided in a sufficient number upon the surface of balloon 144H to cause substance 166 to pass at a predetermined desired rate. Accordingly, pore 120H size and quantity are determined in part based upon the viscosity of substance 166, as well as the pressure of substance 166 within balloon 144H. Substance 166 can be the same substance as described with respect to coating 164, including for example any or all of a lubricious coating, a therapeutic substance, and a substance which can be heated or cooled, as further described above.

Tube 110 can be connected to balloon 144H to extend within balloon 144H as shown in FIG. 12, or tube 110 may terminate at a peripheral surface of balloon 144H, as shown in

FIG. 13. In the example of FIG. 12, tube 110 can be used to push balloon 144H along the length of the esophagus, and to maintain balloon 144H in a desired deployment orientation extending at least along tube 110.

Balloon 144H can be inserted into the esophagus in a deflated, partially inflated, or fully inflated state. If not fully inflated, a gas or fluid can be used to inflate balloon 144H to a desired pressure once balloon 144H is in a desired position within the esophagus. The desired or optimal pressure can be chosen to achieve an estimated or actual: desired final size of balloon 144H; desired pressure of an introduced substance 166; desired internal pressure within balloon 144H; and/or stiffness of the balloon 144H material. While a combination of gas and fluid, different gases, or different fluids can be used to inflate balloon 144H, inflation can be carried out solely by introducing substance 166 at a faster rate than substance 166 can pass through pores 120H.

Following inflation, to maintain a predetermined extent of inflation, substance 166 can be introduced at about the same rate that substance 166 is collectively passing out of the balloon through pores 120H. To remove balloon 144H from the body, deflation may be carried out in advance if desired, by reducing the pressure at which the inflation medium is introduced.

Inflation medium can additionally be aspirated out of balloon 144H during deflation, or to discontinue passage of substance 166 through pores 120H into the body. Alternatively, to discontinue passage of substance 166, a material can be introduced into balloon 144H which causes substance 166 to become too viscous to pass through pores 120H, or which will collect at pores 120H to cause blockage of pores 120H. Where a gas is used to generate sufficient pressure within balloon 144H to cause substance 166 to pass through pores 120H, the gas pressure can be reduced below that required to cause such passage.

In an embodiment, the gas introduced into balloon 144H is substance 166. As such, the gas can be heated or cooled, and can include one or more therapeutic gases, for example gases which reduce pain, treat tissue damage, or change pH within the body. In this embodiment, gas emitted through pores 120H substitutes for a desired lubricious property of a substance 166 which is a liquid.

In FIG. 13, device 100H is shown without a rendering of the esophagus, and which contains a core extension 168 A, which elutes or releases a substance as described with respect to extension 168 of FIG. 11. Either core extension 168 or 168A can be linear or spiral shaped, as shown in FIGS. 10 and 13, or each may have any other simple or complex shape. Forming extension 168 A as a spiral facilitates inserting a longer extension, relative to a single linear shape, within balloon 144H.

In a variation of FIGS. 12-13, balloon 144H does not include pores 120H, and substance 164/166 is coated upon an exterior of balloon 144H prior to or subsequent to insertion into the esophagus. Thereafter, when balloon 144H is expanded inside the esophagus, the coating is brought into contact with the esophagus, or is positioned closer to an inner esophageal wall. The coating in this embodiment can be heated or cooled prior to insertion into the esophagus, or a substance 164/166 can be introduced into the esophagus after insertion, either before or after inflation, to be deposited upon an exterior surface of balloon 144H, where it can be cooled or heated previous to or after being so deposited. In such embodiments where pores 120H are not present, a heated or cooled substance can be introduced into an interior of balloon 144H, whereupon an external surface of balloon 144H will become heated or cooled. As such, with or without a coating 164 upon an external surface of balloon 144H, an interior surface of the esophagus can be treated by cooling or heating when balloon 144H is proximate or in contact with such surface.

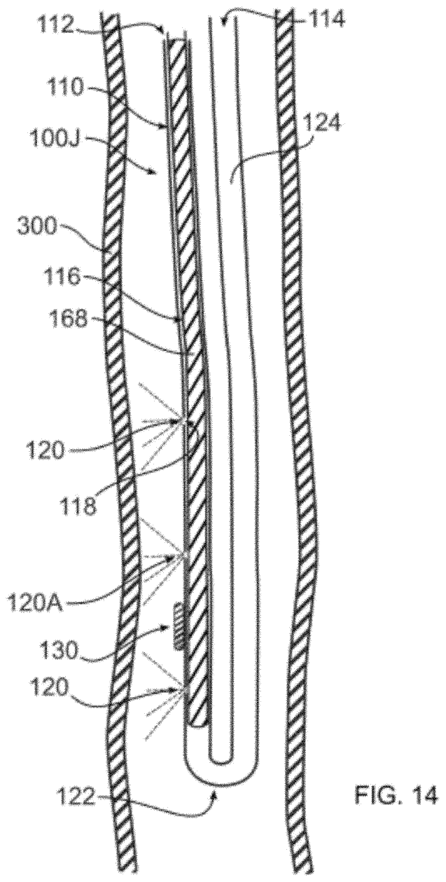

With reference to FIG. 14, device 100J is constructed and used as described with respect to FIG. 1, with certain exceptions. In particular, a core or eluting extension 168 (shown with hatching) is inserted within sleeve 116. When device 100J is inserted into the body, eluting extension 168 is warmed to thereby release a therapeutic substance which protects or prepares the esophagus for the introduction of heat or cold applied during treatment of nearby tissues of the heart, as described herein. The therapeutic substance eluted or released when extension 168 is dissolved can be as described with respect to extension 168 of FIG. 11 or 13, or other therapeutic substance disclosed herein.

In FIG. 14, ports 118 are all aligned simultaneously, so that the eluting substance can migrate through ports 118 of sleeve 116, and ports 120 of tube 110, and out of device 100 J, to be deposited upon an inner surface of the esophagus. While mutually aligned ports 118/120 are shown in FIG. 14, they may be staggered as shown in FIG. 1, for selective opening. Likewise, the ports in FIG. 1 may be aligned as shown in FIG. 14, as elements of the various embodiments herein may be combined or exchanged, as would be understood by one skilled in the art.

Once eluting extension 168 has eluted its therapeutic contents, or has dissolved releasing the therapeutic contents, or has expanded by heat of the body to drive the therapeutic substance through ports 118/120, ports 118 can be displaced by sliding sleeve 116 relative to tube 110, to close the passage through ports 118 and 120. A determination of when a particular therapeutic substance has been sufficiently released can be made, for example, in consideration of an amount of time during which extension 168 is at body temperature, a time elapsed since ports 118/120 were mutually opened, physiological parameters of the patient, or the dissolution of eluting extension 168, either by physically probing sleeve 116, or by indirect measurement, for example by testing an ability to flow a gas or fluid past eluting extension 168. Once ports 118/120 are mutually closed, cooling or heating can be carried out as described herein in a closed loop system as shown in FIG. 14, or by any other closed loop system described herein, for example using an outer tube 144 or 160, as shown in FIG. 8 or 10, and can include a cooling/heating coil as described with respect to FIG. 9.

A process thereby can include any or all of the following steps: (a) introducing device 100 J and eluting extension 168 into the esophagus, (b) aligning ports 118/120, (c) waiting for or otherwise causing release of the therapeutic substance from eluting extension 168, for example by introducing a hydration fluid, or gas pressure, (d) mutually closing ports 118/120 when sufficient therapeutic substance has been released, and (e) introducing a cold or warm liquid or gas into inlet 112 to be received through outlet 114, to be thereby reheated or cooled and reintroduced into the closed system, or to be continuously reintroduced and discarded, and (f) to remove the system once the potential harm to the esophagus is no longer present.

The foregoing process can be carried out by medical personnel, for example by an anesthesiologist, and may be carried out with the assistance of computing technology as described herein. The computing technology can carry out any or all of gathering sensor input, such as that of temperature or pressure sensors, aligning or closing an opening between ports 118/120 or other port system, determining when a release of therapeutic substance is complete or sufficient, and activating pumps or flow associated with closed loop cooling or heating, for example.

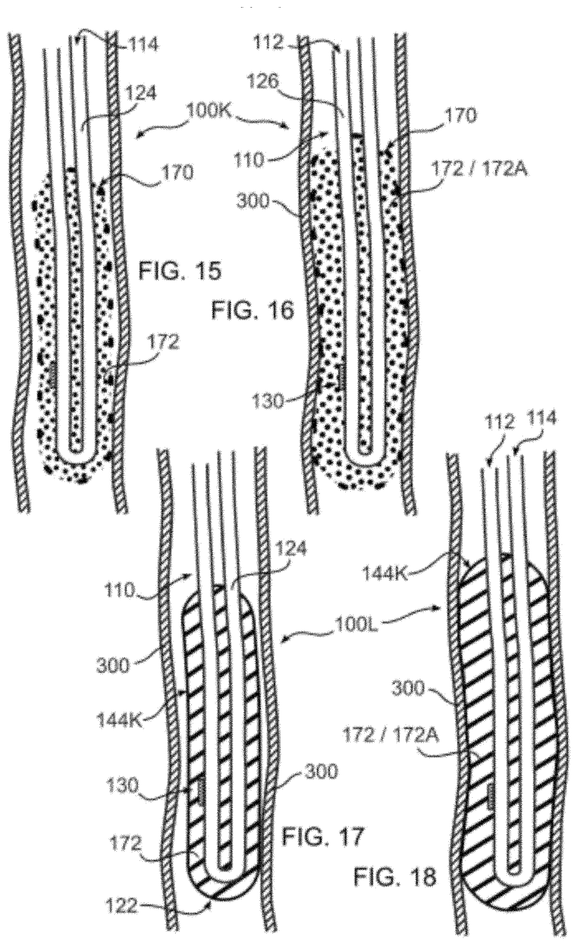

Referring now to FIGS. 15-18, devices 100K-L include an expandable sponge 170 or balloon 144L which contains an expandable biocompatible material 172 which readily transmits heat or cold. Examples of such material include a viscous colloid or gel; a colloid or gel including glycerin; an expandable lattice polymer including for example a low carboxylate acid copolymer; a shape memory expandable polymer, including for example poly(propylene carbonate) (PPC)/polycaprolactone (PCL); expandable polymeric microspheres; or any thermally transferring material which can be expanded once placed at a therapeutic location within the esophagus 300.

In FIGS. 15-16, a sponge 170 or sponge-like material surrounds any of the systems 100 depicted in FIG. 1-7, 9, or 14. In FIGS. 15-16, a simple closed-loop system is depicted as shown in FIG. 7, for clarity, although it should be understood that an open-loop system as shown in the remaining figures can be used. In FIG. 15, sponge 170 is at least partially dried so that it has a smaller than maximum dimension, enabling device 100K to be more easily inserted into the esophagus and positioned at a site of therapy, for example near to the heart. It may be desired to retain some moisture within sponge 170, for example, to ensure that the surface thereof is soft and resilient, to protect body tissue.

As sponge 170 expands, material 172 can elute throughout sponge 170 and escape sponge 170 to contact the inner surface of esophagus 300, improving thermal transfer. Additionally, material 172 can comprise or include a therapeutic substance, for example a healing or antimicrobial agent, which can contact esophagus 300.

As device 100K is inserted, and while it is positioned, sponge 170 can begin to absorb body fluids, and expand to contact inner surfaces of esophagus 300. Cooled or heated fluid can then be circulated through tubes 110 as described elsewhere herein, to transfer or remove heat to or from the sponge. The expandable biocompatible material 172 within sponge 170 then transfers the heat or cold to the inner surface of the esophagus, providing the intended therapeutic benefit. Where an open or openable loop system is employed, liquid introduced into the system can be caused to at least partially escape into sponge 170, thereby accelerating expansion of sponge 170, and can, if desired, introduce additional material 172 into sponge 170. Sensor 130 can be provided, to function as described elsewhere herein and provide data regarding the efficacy of the heating or cooling process.

In an alternative embodiment, material 172A is substituted for material 172, and is a thermally expanding material, whereby when warm fluid is passed through tubes 110, material 172 A expands thereby causing sponge 172 to expand and contact inner surfaces of esophagus 300. Examples of material 172A include thermally activated shape memory polymers (SMPs), and thermally expanding colloids or gels.

Collapse of sponge 170 for withdrawal of system 100K can be achieved by withdrawing fluid 172 when a partially or fully open system is used; allowing sufficient time to lapse for sponge 170 to dry sufficiently, aspirating material from sponge 170, or gently applying pressure to sponge 170, for example during withdrawal, whereby fluid is forced from sponge 170. Where a thermally expanding material 172A is used, cooled fluids can be circulated through tubes 110 to cause contraction of material 172A, or material 172A can otherwise be allowed to cool and contract to facilitate removal of device 100K.

In FIGS. 17-18, a balloon 144L is substituted for sponge 170 in device 100L. As described with respect to device 100K, tubes 110 can be formed as open or closed-loop systems, although a closed-loop system is shown in FIGS. 17-18 for clarity. Additionally, as described with respect to device 100K, material 172 as described above is introduced into balloon 144L either before or after insertion of device 100L. Similar to device 100K, balloon 144L is not fully inflated with material 172, or material 172 is not fully expanded, as device 100L is inserted into and positioned within the esophagus. Once positioned, balloon 144L can be further inflated by introducing a fluid or additionally material 172 using a partial or fully open system 100 of the disclosure, such as are shown in FIG. 1 and FIG. 4, respectively, for example. Once balloon 144L is fully inflated, heating or cooling energy is transferred from the tubes 110, through material 172 and the surface of balloon 144L, to the inner surface of the esophagus.

Where the material is thermally expanding material 172A, heat energy introduced into tubes 110 causes expansion of material 172 A within balloon 144L, and thereby expansion of balloon 144L into contact with the inner surface of esophagus 300, thereby to transfer heat energy to the esophagus.

Deflation of balloon 144L for withdrawal of system 100L can be achieved by withdrawing fluid 172 when a partially or fully open system is used. Alternatively, balloon 144L can be pierced or otherwise torn or opened, for example with a rip cord extending outside of the body, to release material 172. Where a thermally expanding material 172A is used, cooled fluids can be circulated through tubes 110 to cause contraction of material 172 A, or material 172 A can otherwise be allowed to cool and contract to facilitate removal of device 100L.

Alternatively, balloon 144L can include small or microscopic pores which gradually release material 172/172 A, enabling gradual shrinking of balloon 144L. As described above, material 172/172 can comprise or include a therapeutic substance which is beneficial when contacting the esophagus.

In the various embodiments herein, element 168/168 A can be formed together with or as part of steerable element 150. For example, a steerable catheter or alternatively a stylet which is otherwise manipulatable, can be coated with or formed with the colloid, dissolving, or eluting material which releases the therapeutic substance as described herein.

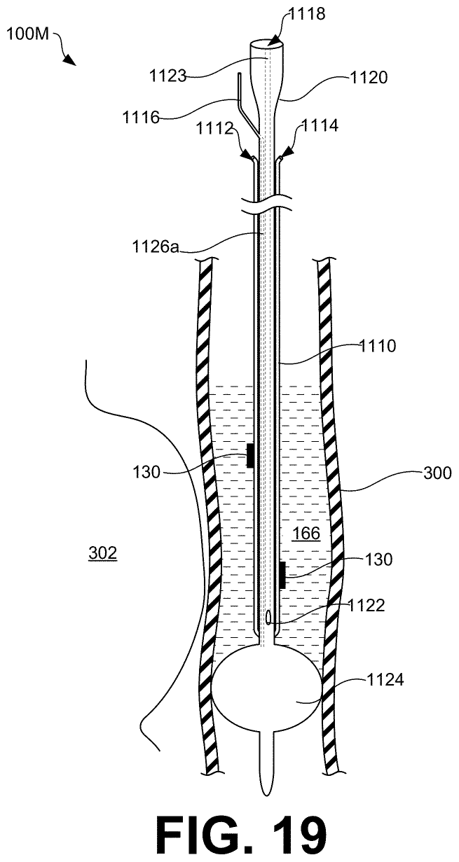

Turning now to FIGS. 19-22, shown are examples of temperature-control devices 100 (e.g., 100M, 100N, 100P, 100Q) for cooling the esophagus during an atrial ablation according to various embodiments of the present disclosure. The temperature-control device 100 includes a catheter 1120 comprising a flexible elongated body that extends from a proximal end to a distal end. The catheter 1120 is configured and sized to be passable from outside of the body to the interior area of the esophagus 300.

According to various embodiments, the temperature control device 100 can comprise a coolant tube 1110 having an inlet 1112 and an outlet 1114 that is disposed around the catheter 1120. The coolant tube 1110 can be made of any biocompatible material that is water impermeable but with adequate thermal conductivity to transfer heat from the system. For example, the coolant tube 1110 can be made of silicone, PVC, natural rubber, styrene butadiene rubber, polyisobutene, polyethylene vinylacetate, ethylene-propylene di-monomer (EPDM), nylons, PET, fluoro-containing co-polymers such as perfluoroethylene-propylene, polypropylene, polyacrylonitrile, polyvinyl alcohol, and/or other type of material as can be appreciated. According to various embodiments, the coolant tube 1110 can be filled with carbon, graphene, and/or metal particles to increase thermal conductivity. In some embodiments, the coolant tube 1110 does not have to be very flexible, and a thin-walled metal could be used as well if it could be bent without kinking.

The inner and outer diameter of the coolant tube 1110 can be selected based on the material used to provide sufficient flow and thermal conductivity. For example, in some embodiments, the coolant tube 1110 can have an outer diameter of about 0.5 to 8.0 mm. As an example, the coolant tube 1110 can have an outer diameter of 1.7 mm and an inner diameter of 0.76 mm. In some embodiments, the coolant inlet is attached to a pump (not shown) configured to pump a heated or cooled fluid through the coolant tube 1110. The fluid preferably has a high specific heat. The fluid can comprise water, saline, and/or any other type of fluid capable of being sacredly ingested such as an emulsion of fat in water that does not damage the material of the device as can be appreciated.

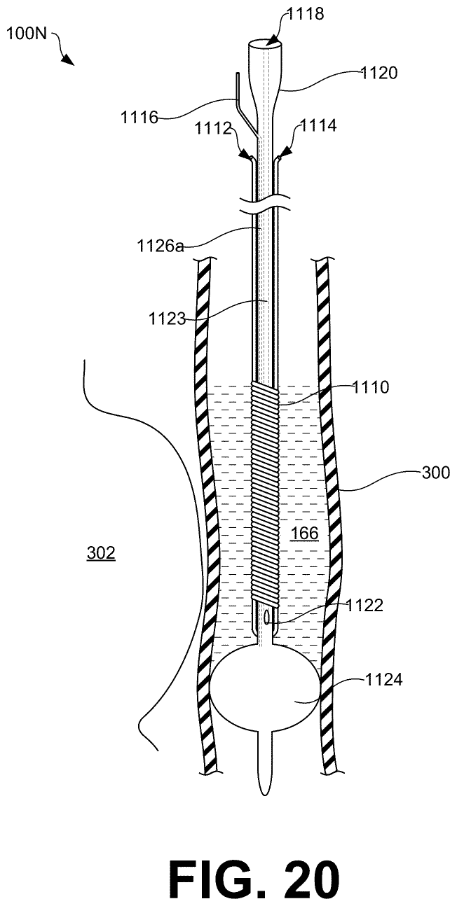

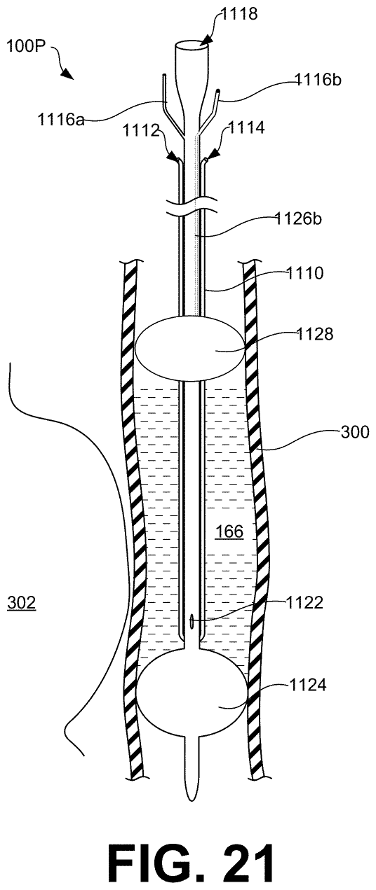

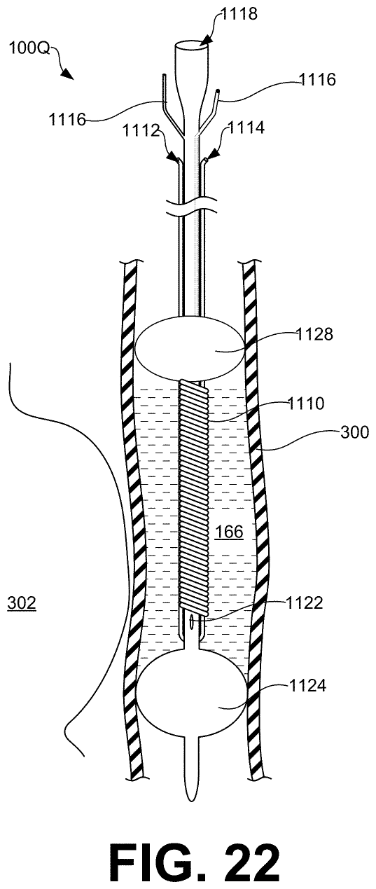

FIGS. 19 and 21 illustrate examples of the coolant tube 1110 extending longitudinally along the length of the catheter 120 with a single bend at the distal end before extending longitudinally back along the catheter and out of the body. In other examples, as shown in FIGS. 20 and 22, the coolant tube 1110 can be coiled with one or more loops around the outer surface of the catheter 1120. Although the coolant tube 1110 in FIGS. 20 and 22 illustrate a coil with multiple loops surrounding the catheter 1120, the coolant tube 1110 can comprise a coil around the catheter 1120 having one or more loops. For example, the coolant tube 1110 can extend towards a distal end of the catheter 1120 and loop at least one time around the catheter 1120 before returning towards the proximal end of the catheter 1120. It should be noted that while the coolant tube 1110 is described as a coil with one or more loops around the catheter 1120 or a tube that runs lengthwise along the catheter 1120, the coolant tube 110 can be formed in any other shape or pattern for optimal surface area as can be appreciated.

The catheter 1120 includes a gel inlet 1118 at its proximal end and a gel port 1122 fluidly connected to the gel inlet 1118 via a gel lumen 1123 extending through the catheter 1120. The gel port 1122 is positioned about the catheter 1120 and configured to release a substance 166 injected in the gel inlet 1118 through the gel lumen 1123 and into the esophagus 300 to serve as a medium for convective heat exchange.

According to various embodiments, as shown in FIGS. 19-22, the catheter 1120 also includes a distal inflatable balloon 1124 at the distal end of the catheter 1120. The distal inflatable balloon 1124 is fluidly connected to an inflation inlet 1116 (e.g., 1116a, 1116b) at the proximal end of the catheter 1120 through a balloon inflation lumen 1126 (e.g., 1126a, 1126b) that extends through the catheter 1120. The inflation inlet 1116 is configured and coupled to the distal balloon 1124 such that injection of an inflation fluid into the inflation inlet 1116 inflates the distal balloon 1124 to a size sufficient to block the esophagus 300 and trap the substance 166 above the distal balloon 1124 to prevent the substance 166 from entering the stomach. The distal balloon 1124 can be in a deflated state during insertion and removal of the temperature-controlling device 100 into an esophagus or other suitable area.

As shown in FIGS. 20 and 22, temperature-controlling device 100 can comprise a proximal balloon 1128 positioned at a proximal portion of the catheter 1120 above the gel port 1122 of the catheter 1120. The proximal balloon 1128 is fluidly connected to an inflation inlet 1116 such that injection of an inflation fluid into the inflation inlet 1116 inflates the proximal balloon 1128 to a size sufficient to block the esophagus 300 and trap the substance 166 below the proximal balloon 1128 to prevent the substance 166 from moving into the lungs. The proximal balloon 1128 can be in a deflated state during insertion and removal of the temperature-controlling device 100 into an esophagus 300 or other suitable area.

According to various embodiments, the proximal balloon 1128 is disposed around an outer surface of the catheter 1120. In some embodiments, the proximal balloon 1128 surrounds the catheter 1120 and at least a portion of the coolant tube 1110 disposed along the catheter 1120. Although shown separately in FIGS. 20 and 22, in some embodiments, the inflation inlet 1116 that is fluidly coupled to the proximal balloon 1128 is the same inflation inlet 1116 that is fluidly coupled to the distal balloon 1124 such that inflation fluid travels through the same balloon inflation lumen 1126 of the catheter 1120. In other embodiments, the inflation inlet 1116 that is fluidly coupled to the proximal balloon 1128 is separate from the inflation inlet 1116 that is fluidly coupled to the distal balloon 1124. For example, the catheter 1120 may comprise a second balloon inflation lumen 1126b that extends through the catheter to an entry point of the proximal balloon 1128. In other embodiments, the inflation inlet 1116 is coupled to a tube (not shown) having a balloon inflation lumen 1125 that is coupled to the proximal balloon 1128 and separate from the catheter 1120.

According to various embodiments, the inflation fluid can comprise air, saline, and/or other types of inflation fluids capable of being sacredly ingested such as an emulsion of fat in water that does not damage the material of the device as can be appreciated. In addition, although the proximal balloon 1128 is described as an inflatable balloon, in some embodiments, the proximal balloon 1128 can comprise an expandable sponge and/or other material that can be used to trap the substance 166 below the proximal balloon 1128, sponge, and/or other suitable component.

For example, although FIGS. 21 and 22 illustrate a proximal balloon 1128, the proximal balloon 1128 can comprise a sponge. According to various embodiments, an expandable sponge for example, can be at least partially dried so that it has a smaller than maximum dimension, enabling device 100 to be more easily inserted into the esophagus 300 and positioned at a site of therapy, for example near to the heart. It may be desired to retain some moisture within sponge, for example, to ensure that the surface thereof is soft and resilient, to protect body tissue. When inserted, the sponge can expand to contact inner surfaces of esophagus 300 and trap the substance 166 below the sponge. In some embodiments, cooled or heated fluid can then be circulated through tubes 1110 as described elsewhere herein, to transfer or remove heat to or from the sponge or proximal balloon 1128. In some embodiment, expandable biocompatible material within a sponge can then transfer the heat or cold to the inner surface of the esophagus 300, providing the intended therapeutic benefit.

According to various embodiments, the device 100 can comprise a temperature sensor 130. As shown in FIG. 19, a temperature sensor 130 can be positioned at one or more locations along catheter 1120. This sensor 130 can transmit temperature data corresponding to an adjacent area along the esophagus 300. A plurality of sensors 130 can provide temperature information for a plurality of areas along esophagus 300, whereby signal processing equipment connected to sensors 130 can identify particular areas of the esophagus which are experiencing or anticipated to experience an undesired temperature change. Sensors 130 can transmit this data via wires, or by a wireless communication, such as WIFI, BLUETOOTH or other nearfield protocol, and any other wireless protocol. To avoid inadvertent heating of sensor by radiofrequency associated with RF ablation, it can be advantageous to avoid using metal in sensor. This may be achieved using a fiber optic sensor, for example. Other non-metallic or metallic temperature sensor technologies may be used for sensor.

In an embodiment, an electronic processor 802 receives temperature information from sensors 130, and reports elevated temperatures. In some cases, the electronic processor 802 controls rate of flow through the coolant tube 1110.

In some embodiments, the device 100 further includes a steerable element (not shown) inserted into an interior of the catheter 1120, the steerable element configured to be bent when positioned inside the body and in the interior of the catheter 1120 to thereby cause a change in an orientation of the catheter 1120 within the body.

As discussed above, in certain aspects, the therapeutic substance 166 discussed herein can include one or more gels that can be readily cooled or heated as needed. The gels are formulated such that they can be injected into the esophagus via the devices 100 as described herein. The gels are composed of water and a non-toxic polymeric material suitable for administration to a subject.

The selection of the polymeric material can vary. In one aspect, the polymeric material is a polyalkylene glycol. “Polyalkylene glycol” as used herein refers to a condensation polymer of ethylene oxide or propylene oxide and water. Polyalkylene glycols are typically colorless liquids with high molecular weights and are soluble in water as well as some organic solvents. In one aspect, the polyalkylene glycol is polyethylene glycol and/or polypropylene glycol. In another aspect, the polyalkylene glycol is monomethoxy polyethylene glycol. In one aspect, the polyalkylene glycol is Miralax© (polyethylene glycol having an average molecular weight of 3,350 manufactured by Bayer) or Carbowax™ (polyethylene glycol having an average molecular weight of 600 to 6,000 manufactured by Dow Chemical).

In one aspect, the polyalkylene glycol is a poloxamer. Poloxamers are nonionic triblock copolymers composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 Da to 15,000 Da, 3,000 Da to 14,000 Da, or 4,000 Da to 12,000 Da. Poloxamers useful herein are sold under the tradename Pluronic® manufactured by BASF.

When a polyalkylene glycol is used to produce the substance 166, the substance 166 has a low dielectric constant. In one aspect, the dielectric constant of the substance 166 less than 20, or is about 10, about 11, about 12, about 13, about 14, about 15, about 16, about 17, about 18, about 19, or less than about 20, where any value can be a lower or upper endpoint of a range (e.g., about 11 to about 18, about 13 to about 16, etc.) as measured with a TR-1A Ratio Arm Transformer Bridge from Ando Electric Co. In another aspect, the polyalkylene glycol has a molecular weight of about 600 Da to about 6,000 Da, or about 600 Da, about 750 Da, about 1,000 Da, about 1,500 Da, about 2,500 Da, about 3,000 Da, about 3,500 Da, about 4,000 Da, about 4,500 Da, about 5,000 Da, about 5,500 Da, about 6,000 Da, where any value can be a lower or upper endpoint of a range (e.g., about 600 Da to about 2,000 Da, about 3,000 Da to about 5,000 Da, etc.). However, it should be noted molecular weights greater than 6,000 Da for the polyalkylene glycol can be used as can be appreciated. According to various embodiments, the pure polyethylene polymers have dielectric constants of about 20, and their solutions will be some average with that of water.

The substance 166 can be prepared by admixing the polymeric material in water with one or more optional components as needed. The admixing of the polymeric material with water can be conducted at room temperature or at elevated temperatures depending upon the selection and amount of polymeric material used. In one aspect, the gel has a viscosity high enough to allow the inflated balloon to be immobilize it in the esophagus, but low enough to allow it to be injected and aspirated. The amount of polymeric material used to produce the gel can be modified in order to fine-tune the viscosity of the gel. In one aspect, the polymeric material is from about 0.1 wt % to 5 wt % of the gel, or is about 0.1 wt %, about 0.5 wt %, about 1.0 wt %, about 1.5 wt %, about 2.0 wt %, about 2.5 wt %, about 3.0 wt %, about 3.5 wt %, about 4.0 wt %, about 4.5 wt %, or about 5.0 wt %, where any value can be a lower or upper endpoint of a range (e.g., about 0.1 wt % to about 4.0 wt %, about 0.5 wt % to about 2.0 wt %, etc.).

In certain aspects, the substance 166 can be produced when it is time to use the device described herein. In one aspect, a kit comprising the device 100 described herein can include the components to produce the substance 166. For example, the polymeric material can be provided as a dry material in the kit with instructions for admixing the polymeric material with a certain volume of water. In other aspects, the kit can include the device 100 with the substance 166 already prepared for use. The kits described herein can also include one or more syringes for injecting the gel into the devices described herein.

Features of the various embodiments herein may be combined or substituted. Non-limiting illustrative examples include: the use of the sliding port closing system of FIG. 1 or 1A with other supply tubes, such as within tube 110 of FIGS. 10-13, to control the location and extent of discharge of substance 164; the use of core extension 168 within any embodiment; the use of coil 152 within tube 160 or balloon 144; the use of steerable device 150 with any embodiment; the use of external ports to deposit substance 166 upon balloon 144H; sensors with any embodiment; the use of substance 166 to coat or be released by any embodiment, and/or the use of proximal balloons 1128 and/or distal balloons 1124 (FIGS. 19-22) to trap the substance 166 in the esophagaus.

Devices 100 of the disclosure can be used in the various manners described herein, and can additionally be advantageously used in outflow tract tachycardia or right ventricle ablations in the epicardium, particularly where the endocardium is thin. Additionally, devices 100 can be used to buffer the convective heat introduced from an ablation catheter, enabling in certain cases transmittal of full thickness lesions with a lower chance of perforation or collateral damage to adjacent epicardial arteries than in cases where devices 100 are not used.

Devices 100 of the disclosure can have any size which can be effectively inserted into the esophagus of a given patient, which varies widely according to human anatomy. An example non-limiting range of diameter includes 4 mm to 20 mm, and lengths of 250 to 500 cm. Smaller, wider, longer, or shorter sizes can be used depending upon the patient size, whether or not it is desired for the cooling/warming area of the device to contact the esophageal wall, and a length extending outside of the body that is convenient. Appropriate biocompatible materials can be used, as understood within the art, although the avoidance of metal is advantageous to avoid undesired retransmission of RF energy within the esophagus. Flexible components such as tube/sleeve 116 and housing/tube 110 are advantageously made with a biocompatible polymer with sufficient flexibility, durability, and lubricity, as would be understood within the art.

Examples can include Poly(ethylene) (PE) (HDPE, UHMWPE); Poly(propylene) (PP); Poly(tetrafluroethylene) (PTFE) (Teflon), extended-PTFE; Ethylene-co-vinylacetate (EVA); Poly(dimethylsiloxane) (PDMS); Poly(ether-urethanes) (PU); Polyethylene terphthalate) (PET); and Poly(sulphone) (PS), although other materials, including polymeric, synthetic, and natural, can be used.

### EXAMPLES

### Example 1: Development of in Vitro Testing Platform

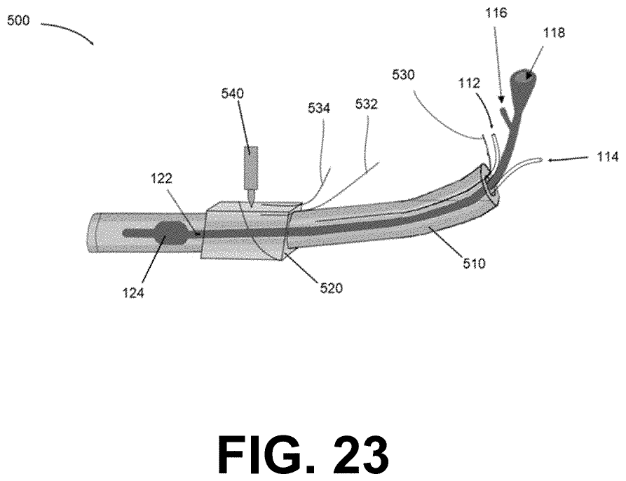

An in vitro testing stand for prototype testing was developed with the goal of mimicking the spatial relationship and thermal conductivity of the left atrium and esophagus. The esophagus was represented by a flexible polyvinyl alcohol (PVA) foam tube, with an inner diameter of about 2 cm and a thickness of about 5 mm. The inside of the PVA tube was coated with a silicone sealant to limit the porosity of the tube. A 5% agar hydrogel with an outer thickness of 5 mm was used as a phantom of the left atrial tissue. Heat was applied using a soldering iron, and the entire system was submerged in a 37° C. 0.7% saline water bath. The agar tissue phantom was submerged 1 mm below the surface of the saline to mimic surface flow of blood while also reducing heat loss at the ablation site. Tests were performed to compare the agar tissue phantom and soldering iron heat application with previously developed in vitro models that utilized ablation catheters to validate the testing platform. The final rendition of the testing platform is shown in FIG. 23.

### Example 2: Development and Testing of Prototypes

A prototype design (FIGS. 19 and 20) was created that utilizes the balloon of a Foley catheter as a mechanism for blocking the flow of a viscous liquid down the esophagus. The Foley catheter was modified by blocking the lowest port and creating a new port above the balloon. Silicone tubing was attached to the catheter to transport room temperature water through the device, with one inlet and one outlet. During use, the catheter is inserted into the esophagus model and the balloon is positioned below the ablation site to avoid pushing the esophagus towards the left atrium. The balloon is then inflated with 5 mL of air to block the esophagus and secure the position of the device. Once the prototype is in place, the inlet tube for room temperature water is attached to a pump and the outlet tube is placed in a waste beaker. This pump system can be replaced with a peristaltic pump. Following the establishment of fluid flow through the silicone tubing, 8 mL of a viscous liquid (alginate, xanthan gum, etc.) at room temperature is delivered through the large port of the Foley catheter using a syringe. The purpose of the viscous liquid is to serve as the medium for convective heat exchange and to remain above the inflated balloon.

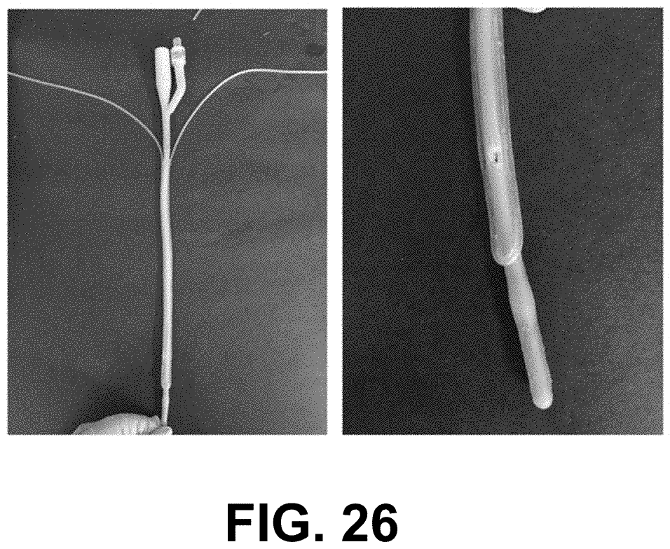

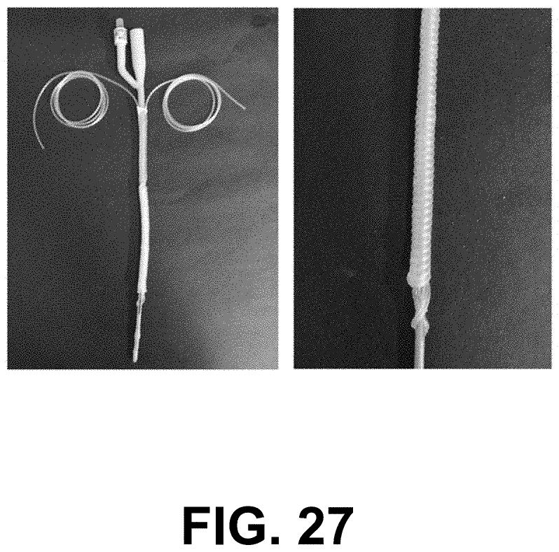

Experiments were performed to determine the proper size and configuration of the silicone tubing for optimum convection prompted by the room temperature water circulation. FIG. 19 shows a one-turn tubing configuration. Experiments were performed to compare changes in temperature 5 mm below the surface of the agar tissue phantom (representative of the esophageal wall) using silicone tubing with a 1.2 mm outer diameter or a 1.7 mm outer diameter in either a one-turn (FIGS. 19 and 26) or coiled (FIGS. 20 and 27) configuration. Based on experimental results, the 1.7 mm outer diameter tubing was selected. The larger tubing size was demonstrated to decrease heating 5 mm below the ablation site when compared to the smaller size tubing. This is thought to be due to the increased flow rate in the 1.7 mm tubing. There was no significant difference observed between the one-turn and coiled conformations following preliminary testing, but the coiled conformation was ultimately selected due to its ability to create a more uniform area of convection within the esophagus.

The 1.7 mm OD coiled silicone tubing prototype has a diameter of 0.85 cm (25.5 F) at the point of largest width.

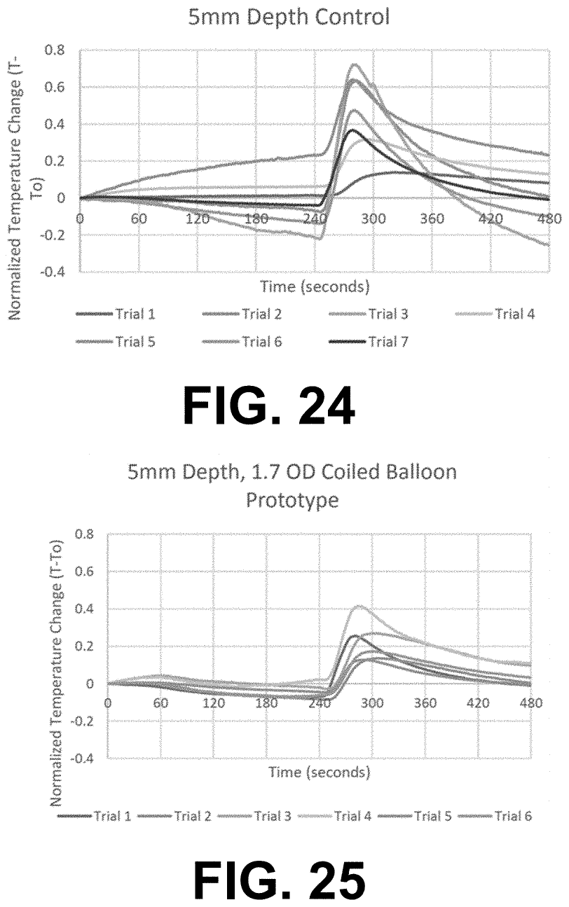

FIGS. 24 and 25 show results comparing the 1.7 mm OD coiled balloon prototype (FIG. 27) to a control (FIG. 26). During these experiments, heat was applied for 30 seconds with a soldering iron at 150° C. at 240 seconds. Temperatures at the site of heat application, 5 mm below the site of heat application, and within the PVA esophagus model were taken over time. The results shown are indicative of the temperature change at the 5 mm depth over time, normalized by the temperature change at the surface. This normalization is performed to account for differences in surface heating due to differences in water height above the site of heat application or differences in heat application using the soldering iron.

The balloon prototype resulted in decreased temperature changes 5 mm below the ablation site, which in this experimental platform is used to indicate the esophageal wall. The data is normalized by the increase in temperature at the ablation site, so the in vitro model asserts that at equal ablation site temperatures, the increase in temperature at the esophageal wall would be lower when the prototype is in use. Specifically, the average maximum temperature at the 5 mm depth for the prototypes were an average of 1.2° C. lower than that of the controls, with average maximum temperatures of 36.8° C. and 38° C. respectively.

One potential modification of this prototype would be to replace the closed loop convective flow of room temperature water with an open loop flow of the viscous fluid. In this case, the bulk viscous liquid would be replaced over time, potentially increasing the convective heat transfer.

Candidates for the viscous liquid included xanthan gum, alginate, and gelatin between 0.5% and 2% concentrations. Gelatin was ultimately excluded due to a phase change from a gel to a liquid around 27° C. Xanthan gum has been the most widely used in these studies due to ease of preparation (including the experiments in FIG. 6). Xanthan gum can be incorporated into water by stirring alone, while alginate requires heating to allow the polymer to degrade. Experiments are still being performed with 1 and 2% concentrations of xanthan gum and alginate to determine any differences.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein.

Example Computing Components

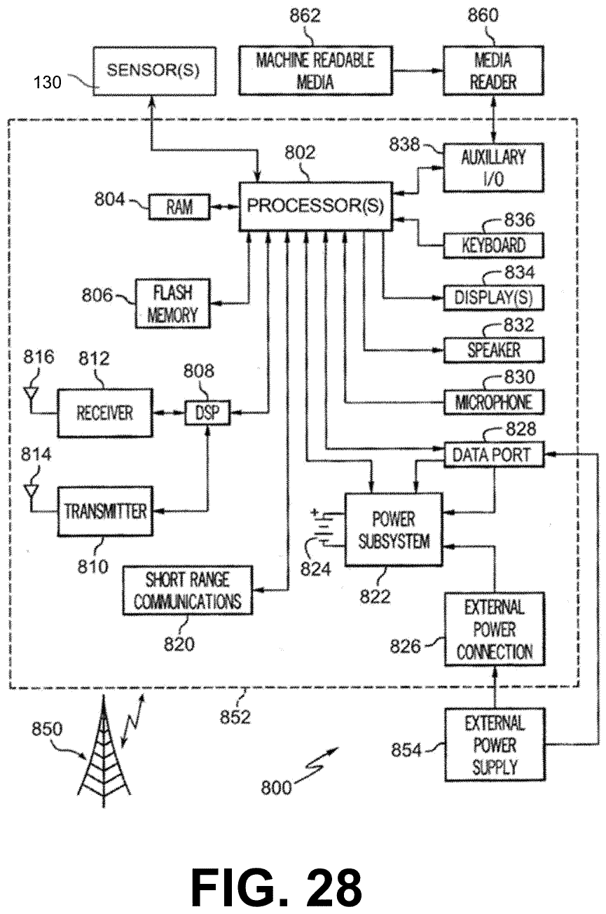

FIG. 28 is a block diagram of an electronic device and associated components 800, which can be used in carrying out the disclosure. In this example, an electronic device 852 is a wireless two-way communication device with voice and data communication capabilities. Such electronic devices communicate with a wireless voice or data network 850 using a suitable wireless communications protocol. Wireless voice communications are performed using either an analog or digital wireless communication channel. Data communications allow the electronic device 852 to communicate with other computer systems via the Internet. Examples of electronic devices that are able to incorporate the above described systems and methods include, for example, a data messaging device, a two-way pager, a cellular telephone with data messaging capabilities, a wireless Internet appliance or a data communication device that may or may not include telephony capabilities. Electronic device 800 can be used, for example, to gather electronic data from sensors 130 by wired or wireless means, to display such data or otherwise communicate such data to medical practitioners, and to control flow of cool or warm fluid through device 100.

The illustrated electronic device 852 is an example electronic device that includes two-way wireless communications functions. Such electronic devices incorporate communication subsystem elements such as a wireless transmitter 810, a wireless receiver 812, and associated components such as one or more antenna elements 814 and 816. A digital signal processor (DSP) 808 performs processing to extract data from received wireless signals and to generate signals to be transmitted. The particular design of the communication subsystem is dependent upon the communication network and associated wireless communications protocols with which the device is intended to operate.

The electronic device 852 includes a microprocessor 802 that controls the overall operation of the electronic device 852. The microprocessor 802 interacts with the above described communications subsystem elements and also interacts with other device subsystems such as flash memory 806, random access memory (RAM) 804, auxiliary input/output (I/O) device 838, data port 828, display 834, keyboard 836, speaker 832, microphone 830, a short-range communications subsystem 820, a power subsystem 822, and any other device subsystems.

A battery 824 is connected to a power subsystem 822 to provide power to the circuits of the electronic device 852. The power subsystem 822 includes power distribution circuitry for providing power to the electronic device 852 and also contains battery charging circuitry to manage recharging the battery 824. The power subsystem 822 includes a battery monitoring circuit that is operable to provide a status of one or more battery status indicators, such as remaining capacity, temperature, voltage, electrical current consumption, and the like, to various components of the electronic device 852.

The data port 828 of one example is a receptacle connector 104 or a connector that to which an electrical and optical data communications circuit connector (not shown) engages and mates, as described above. The data port 828 is able to support data communications between the electronic device 852 and other devices through various modes of data communications, such as high speed data transfers over an optical communications circuits or over electrical data communications circuits such as a USB connection incorporated into the data port 828 of some examples. Data port 828 is able to support communications with, for example, an external computer or other device.

Data communication through data port 828 enables a user to set preferences through the external device or through a software application and extends the capabilities of the device by enabling information or software exchange through direct connections between the electronic device 852 and external data sources rather than via a wireless data communication network. In addition to data communication, the data port 828 provides power to the power subsystem 822 to charge the battery 824 or to supply power to the electronic circuits, such as microprocessor 802, of the electronic device 852.

Operating system software used by the microprocessor 802 is stored in flash memory 806. Further examples are able to use a battery backed-up RAM or other non-volatile storage data elements to store operating systems, other executable programs, or both. The operating system software, device application software, or parts thereof, are able to be temporarily loaded into volatile data storage such as RAM 804. Data received via wireless communication signals or through wired communications are also able to be stored to RAM 804.

The microprocessor 802, in addition to its operating system functions, is able to execute software applications on the electronic device 852. A predetermined set of applications that control basic device operations, including at least data and voice communication applications, is able to be installed on the electronic device 852 during manufacture. Examples of applications that are able to be loaded onto the device may be a personal information manager (PIM) application having the ability to organize and manage data items relating to the device user, such as, but not limited to, e-mail, calendar events, voice mails, appointments, and task items.

Further applications may also be loaded onto the electronic device 852 through, for example, the wireless network 850, an auxiliary I/O device 838, Data port 828, short-range communications subsystem 820, or any combination of these interfaces. Such applications are then able to be installed by a user in the RAM 804 or a non-volatile store for execution by the microprocessor 802.

In a data communication mode, a received signal such as a text message or web page download is processed by the communication subsystem, including wireless receiver 812 and wireless transmitter 810, and communicated data is provided the microprocessor 802, which is able to further process the received data for output to the display 834, or alternatively, to an auxiliary I/O device 838 or the Data port 828. A user of the electronic device 852 may also compose data items, such as e-mail messages, using the keyboard 836, which is able to include a complete alphanumeric keyboard or a telephone-type keypad, in conjunction with the display 834 and possibly an auxiliary I/O device 838. Such composed items are then able to be transmitted over a communication network through the communication subsystem.

For voice communications, overall operation of the electronic device 852 is substantially similar, except that received signals are generally provided to a speaker 832 and signals for transmission are generally produced by a microphone 830. Alternative voice or audio I/O subsystems, such as a voice message recording subsystem, may also be implemented on the electronic device 852. Although voice or audio signal output is generally accomplished primarily through the speaker 832, the display 834 may also be used to provide an indication of the identity of a calling party, the duration of a voice call, or other voice call related information, for example.

Depending on conditions or statuses of the electronic device 852, one or more particular functions associated with a subsystem circuit may be disabled, or an entire subsystem circuit may be disabled. For example, if the battery temperature is low, then voice functions may be disabled, but data communications, such as e-mail, may still be enabled over the communication subsystem.

A short-range communications subsystem 820 provides for data communication between the electronic device 852 and different systems or devices, which need not necessarily be similar devices. For example, the short-range communications subsystem 820 includes an infrared device and associated circuits and components or a Radio Frequency based communication module such as one supporting Bluetooth® communications, to provide for communication with similarly-enabled systems and devices, including the data file transfer communications described above.

A media reader 860 is able to be connected to an auxiliary I/O device 838 to allow, for example, loading computer readable program code of a computer program product into the electronic device 852 for storage into flash memory 806. One example of a media reader 860 is an optical drive such as a CD/DVD drive, which may be used to store data to and read data from a computer readable medium or storage product such as computer readable storage media 862. Examples of suitable computer readable storage media include optical storage media such as a CD or DVD, magnetic media, or any other suitable data storage device. Media reader 860 is alternatively able to be connected to the electronic device through the Data port 828 or computer readable program code is alternatively able to be provided to the electronic device 852 through the wireless network 850.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

Therefore, at least the following is claimed:

1. A device for cooling or warming an interior area of an esophagus during a therapeutic procedure, comprising:
a flexible tube having a proximal end and a distal end, the flexible tube being passable from outside of the body to the interior area of the esophagus and including at least one gel passing port formed through the tube;
a coolant tube affixed to an exterior surface of the flexible tube, the coolant tube extending from the proximal end of the flexible tube to the distal end of the flexible tube; and
at least one balloon affixed to the exterior surface of the flexible tube, the at least one balloon being configured to block the esophagus when inflated to prevent a gel released through the at least one substance passing port from entering another area of the body.

2. The device of claim 1, further including at least one temperature sensor positioned along a length of the tube and configured to output temperature information pertaining to a plurality of areas of the esophagus.

3. The device of claim 1, the tube forming at least one bend whereby the tube is passable back outside of the body, the tube thereby forming two ends both outside of the body, the one or more tube ports positioned proximate the interior area of the esophagus.

4. The device of claim 1, further comprising a flexible sleeve slidable in connection with the tube and including at least one substance passing port formed through the sleeve, the sleeve sized with respect to the tube to form a tight seal with the tube such that when at least one substance passing port of the sleeve is aligned with the at least one substance passing port of the tube, a substance may pass through the sleeve and the tube.

5. The device of claim 4, wherein the sleeve is slidable within the tube.

6. The device of claim 4, wherein the sleeve is slidable along an exterior of the tube.

7. The device of claim 4, wherein a distal end of the flexible tube that is passed first into the body is surrounded by an outer tube which captures liquid which has passed through a flexible sleeve.

8. The device of claim 1, a distal end of the flexible tube that is passed first into the body being surrounded by the at least one balloon which captures liquid which has passed through the flexible sleeve.

9. The device of claim 1, wherein the coolant tube is formed into a coil.

10. A kit comprising the device of claim 1 and the gel.

11. The kit of claim 10, wherein the gel comprises water and a polyalkylene glycol.

12. The kit of claim 11, wherein the polyalkylene glycol comprises polyethylene glycol, polypropylene glycol, monomethoxy polyethylene glycol, a poloxamer, or any combination thereof.

13. The kit of claim 11, wherein the polyalkylene glycol has a molecular weight of about 600 Da to about 6,000 Da.

14. The kit of claim 11, wherein the polyalkylene glycol is from about 0.1 wt % to 5 wt % of the gel.

15. The kit of claim 10, wherein the gel has a dielectric constant of less than 20.

16. The kit of claim 10, wherein the gel comprises a thermally conductive gel.

17. A method for cooling or warming an interior area of the esophagus during a therapeutic procedure comprising:
inserting a temperature-cooling device into the esophagus;
inflating at least one balloon of the device to block at least one section of the esophagus; and
injecting a therapeutic substance into a therapeutic substance lumen of the device in order to deposit the therapeutic substance into the esophagus, the at least one balloon blocking the therapeutic substance from traveling to other areas of the body.

18. The method of claim 17, wherein the therapeutic substance comprises water and a polyalkylene glycol and the polyalkylene glycol comprises polyethylene glycol, polypropylene glycol, monomethoxy polyethylene glycol, a poloxamer, or any combination thereof.

19. The method of claim 18, wherein the polyalkylene glycol has a molecular weight of about 600 Da to about 6,000 Da.

20. The method of claim 18, wherein the polyalkylene glycol is from about 0.1 wt % to 5 wt % of the gel.
